(19) United States
(12) Patent Application Publication
(10) Pub. No.: US 2025/0222195 A1
(43) Pub. Date: Jul. 10, 2025

(54) A CONNECTOR FOR AN IRRIGATION SYSTEM

(71) Applicant: Coloplast A/S, Humlebaek (DK)

(72) Inventor: Lars Stendevad Windeballe, Virum (DK)

(21) Appl. No.: 18/853,441

(22) PCT Filed: Mar. 29, 2023
(86) PCT No.: PCT/DK2023/050065

§ 371 Date: Oct. 2, 2024

(30) Foreign Application Priority Data
Apr. 8, 2022 (DK) PA202270187

(51) Int. Cl. A61M 3/02; A61F 5/442

(52) U.S. Cl. CPC A61M 3/0245; A61F 5/442; A61M 3/0258; A61M 3/0262; A61M 2205/0288; A61M 2210/1067

(57) ABSTRACT

A system for anal or stomal irrigation is described. The system includes a container, tube(s), an anal probe and a pump. The system further includes a connector, connecting the tube(s) to the container.

The invention relates to a connector for use in an irrigation system, in particular a connector for use in a container for an irrigation system.

### BRIEF DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an anal irrigation system as described herein.

FIGS. 2 to 4 illustrate a cross-sectional view of an example of a connector in a position, where it is to be attached (FIG. 2), in an attached or locked position (FIG. 3) and finally in a release position in which it can be removed (FIG. 4).

FIGS. 5 to 7 illustrate a cross-sectional view of an example of a connector in a position, where it is to be attached (FIG. 5), in an attached or locked position (FIG. 6) and finally in a release position which it can be removed (FIG. 7).

FIGS. 8 and 9 illustrate a part of a surface of a container with a guide for a slider configured to release a connector

### DETAILED DESCRIPTION

Examples relate to an anal or stomal irrigation system comprising
a container configured for containing irrigation liquid and configured for being pressurized,
a pump configured for pumping air into the container so as to displace the liquid from the container,
a connector configured for connecting a tube to the container
wherein the connector cooperates with a coupling in the container, the connector being configured to be in two positions with respect to the coupling
a first connected position,
a second disconnected position,
   and wherein the coupling comprises a toggle arm comprising a gripper configured for gripping a flange on the connector, the toggle arm further comprising a first hold which is configured to be in a magnetic hold with a second hold, and thereby hold the connector in a locked position, the toggle arm being tiltably attached to the coupling, such that it is able to tilt between a first position, in which the toggle arm keeps the connector in the first connected position and a second position, in which the toggle arm allows the connector to be in a second disconnected position.

Examples relate to a method of connecting a connector to a container, the container comprising a toggle arm comprising a gripper configured for gripping a flange on the connector, the toggle arm further comprising a first hold which is configured to be in a magnetic hold with a second hold, and thereby hold the connector in a locked position, the toggle arm being tiltably attached to the container, such that it is able to tilt between a first position, in which the toggle arm keeps the connector in the first connected position and a second position, in which the toggle arm allows the connector to be in a second disconnected position, wherein the method comprises the connector being displaced into the container and thus tilting the toggle arm into the first connected position.

Examples relate to a method of releasing a connector from a container, the container comprising a toggle arm comprising a gripper configured for gripping a flange on the connector, the toggle arm further comprising a first hold which is configured to be in a magnetic hold with a second hold, and thereby hold the connector in a locked position, the toggle arm being tiltably attached to the container, such that it is able to tilt between a first position, in which the toggle arm keeps the connector in the first connected position and a second position, in which the toggle arm allows the connector to be in a second disconnected position, wherein the method comprises a magnet being moved over the container such that the toggle arm is tilted into its second position.

Users of anal or stomal irrigation systems sometimes have poor hand dexterity, and they may find it challenging to connect and de-connect a connector from the container or reservoir. Users with poor hand dexterity may have a reduced or lack pinch grip ability. It is therefore an advantage to provide a system and methods, in which the user does not have to rely on pinch grip ability to be able to connect and release a connector from the container. In systems and methods described herein, the user will be able to connect the connector by simply pushing it into the container. When the connector is pushed into the container, the toggle arm mechanism in cooperation with the magnetic hold and the gripping of the connector by the gripper will be able to keep the connector safely connected to the container. When the user wants to release or de-connect the connector again, the user simply moves a magnet over the container and is thus able to get the toggle arm to release its grip on the connector and thereby release and lift the connector outwards from the container.

In the following, whenever referring to a proximal end of end element described herein, the referral is to the end closest to the user of the system. Whenever referring to the distal end of an element, the referral is to the end opposite of that. In other words, the proximal end is the end outwards of the container and the distal end is the end inwards in the container.

The longitudinal direction is the direction from the distal to the proximal end. This may also be called the axial direction for the connector, coupling and dip-tube. The axial direction corresponds to the direction of flow through the connector from the container and outwards through the lumen of the connector. A direction transversely to the axial direction is defined as being a transverse direction.

An irrigation system typically comprises a reservoir or container for irrigation liquid, an anal probe and tubing connecting those two. The system may also include a pump for pumping the irrigation liquid into the intestines.

The anal probe comprises a stem in the form of a tubular part extending from the distal end towards the proximal end. The tip portion is positioned in the proximal end of the stem. The probe may comprise a connector in the distal end of the stem.

The anal probe may comprise retention means, for example in the form of an inflatable balloon or in the form of a foam element.

The connector has two portions, a distal portion, which is configured to be inserted into and removed from the container and a proximal portion configured for providing an interface for connecting a tube to the connector—either by welding or by detachably attaching a tube to the connector. The two portions may be separated by a flange providing an interface between the connector and a surface portion of the container (for example, a lid). The connector has an axially extending lumen configured for liquid flowing through this lumen.

The coupling is positioned in a portion of the container, which is close to a surface, typically a top surface of the container, but it could also be a side-facing surface. The coupling may be integrated in the container such that it is integrally moulded with the container. The coupling may also be an element separate from the container and which is inserted into the container during manufacturing. The connector cooperates with the toggle arm of the coupling to provide a first connected position and a second disconnected position. The coupling further comprises an axially extending lumen in which the distal portion of the connector can be received and through which liquid can flow.

The toggle arm of this disclosure comprises a gripper in a proximal end. Furthermore, the toggle arm comprises a first hold. The toggle arm comprises a distal end. The toggle arm is configured to rotate around a rotational axis, or at least to toggle around a tilting axis, between a first position, in which the connector is freely movable upwards, and a second position, in which the gripper locks the connector to the container. In the illustrated examples, the toggle arm extends perpendicularly to the axial direction of the connector in a first connected position of the connector and is at an angle to the axial direction of the connector in a second disconnected position of the connector.

The first hold at the toggle arm is configured to be in a magnetic hold with a second hold, and thereby hold the connector in a locked position. By magnetic hold is meant that the first hold and the second hold magnetically attract each other. One example is that the first hold is in the form of a magnetically poled material and the second hold is in the form of a magnetic material. Alternatively, the second hold is in the form of a magnetically poled material and the first hold is in the form of a magnetic material. A third option is that the first hold and the second hold are in the form of magnetically poled materials and where the first hold and the second hold are oppositely poled, such that they attract each other.

The magnetic hold should be strong enough to overcome the force on the connector due to the pressure that may be built up inside the container and due to the friction force by liquid passing through the connector in an outwards direction. Furthermore, it is an advantage, if there is a clear tactile feedback to the user, when the connector reaches the first connected position.

In one example, a third magnet is positioned in a slider, which is configured to be slid over a surface of the container to a release position, where it is able to release the magnetic hold between the first hold and the second hold.

Examples relate to the releasing being done as a result of magnetic repulsion between the third magnet and the first hold.

Examples relate to the release being done as a result of magnetic attraction between the third magnet and a release hold positioned at the toggle arm.

Examples relate to the container comprising a guide configured for receiving the slider, such that the slider is easily guided to the correct release position.

Examples relate to the guide being in the form of a curved ridge. Examples relate to the guide being in the form of two curved ridges.

Examples relate to the first hold being positioned at a distal portion of the toggle arm.

Examples relate to the first hold being positioned at a proximal portion of the toggle arm.

Examples relate to the toggle arm being configured for rotating about a rotation point located approximately at a middle portion of the toggle arm.

Examples relate to the toggle arm being configured for rotating about a rotation point located at a distal end of the toggle arm.

Examples relate to the interface between the connector and the coupling including a sealing.

Examples relate to the sealing being in the form of a gasket.

Examples relate to the sealing being in the form of a radially outwards extending bulge on the connector cooperating with a radially inwards extending protrusion on the coupling.

Examples relate to the coupling comprising an axially extending lumen configured to receive the connector. Examples relate to the axially extending lumen having a quadrangular cross-section, e.g. square or rectangular. Examples relate to the axially extending lumen having an oval cross-section, such as elliptical or circular.

Examples relate to the container comprising a dip-tube configured for providing flow of liquid from a bottom portion of the container to the lumen of the coupling.

Examples relate to the dip-tube extending to the bottom of the container. In these examples a distal portion of the dip-tube, or at least a distal end of the dip-tube, is at the bottom of the container. This makes it possible to use all the liquid in the container, because all of the liquid can flow to the coupling and connector and from there through tubing to the anal probe.

Examples relate to the dip-tube being integral with the coupling, such that the coupling is formed in a proximal portion of the dip-tube.

Examples relate to the dip-tube being attachable to the coupling. It may be an advantage that the dip-tube can be exchanged separately from the coupling, in particular, if the coupling is integrated into the container in such a way that it is difficult to replace.

Examples relate to the system comprising a pump configured to displace the liquid from the reservoir.

Examples relate to the pump being an electric pump. An electric pump may be an advantage for users with poor hand dexterity or low strength in their hands because it may be easier for those users to operate an electric pump.

Examples relate to the pump being a manual pump. Such a system may be an advantage for users who have to take the system with them, because operation of a hand pump can be done anywhere and does not require any battery power or electrics.

Examples relate to the pump being a liquid pump. A liquid pump is configured for directly displacing the liquid and does not require a container, which can be pressurized.

Examples relate to the pump being an air pump configured to pressurize the container and thereby displace the liquid. An air pump is not in contact with the liquid and does not have to be cleaned or have parts replaced due to having been in contact with liquid. It is easier to reuse over a long period of time.

### Detailed Description of the Drawing

Initially, it shall be noted that the figures are schematic illustrations intended only to address the principles and functions of the anal probe described herein and are not to be considered limiting to the scope of the attached claims. Furthermore, the figures and particularly the individually illustrated elements are not necessarily to scale, neither individually nor in relation to each other.

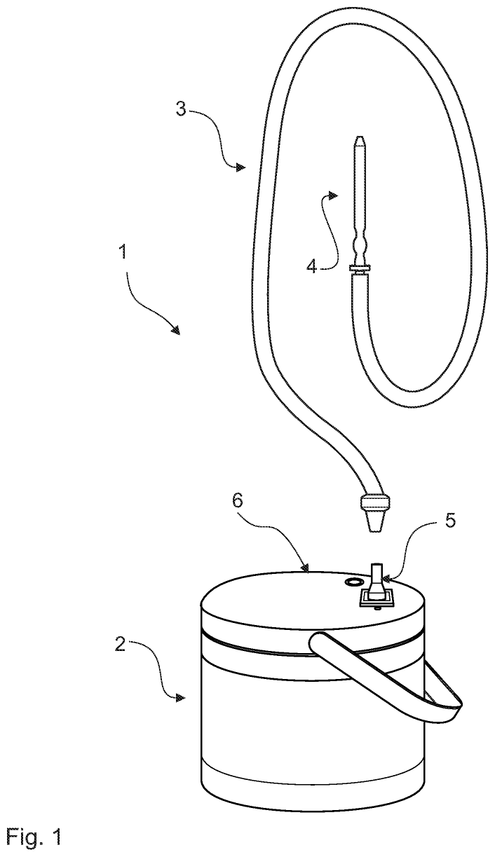

FIG. 1 illustrates an anal irrigation system 1 as described herein. The system comprises a container for irrigation liquid 2, a tube 3 connected to an anal probe 4. A connector 5 connects the container 2 to the tube 3. In the example of the system illustrated in FIG. 1, the connector 5 is positioned in the lid 6 of the container. The container 2 may have other features, such as a handle, a scale, and so forth.

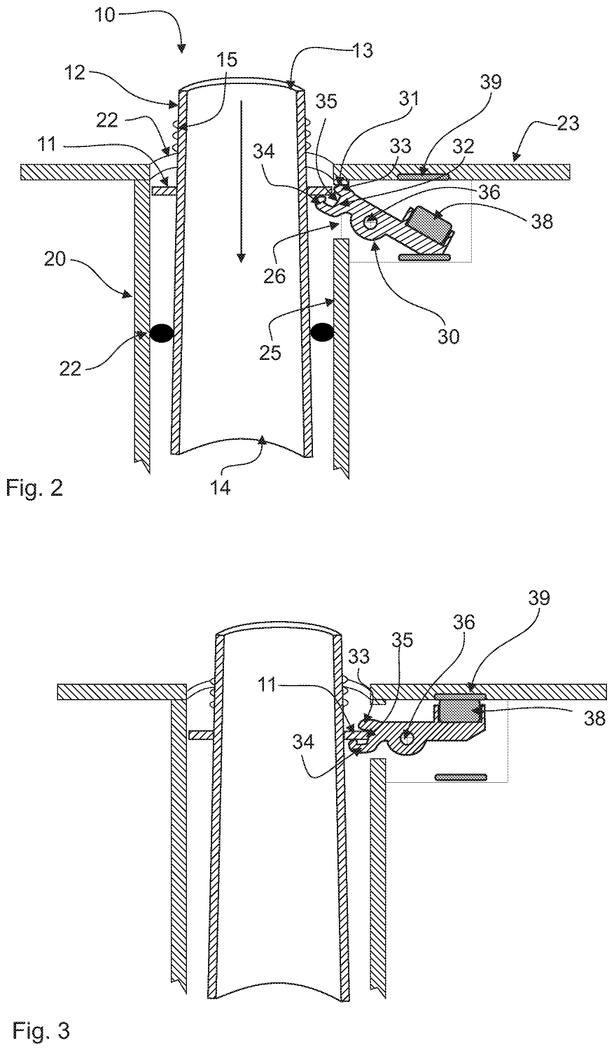

FIGS. 2 to 4 illustrate an example of a connector and coupling as described herein.

The connector 10 is provided with a flange 11, positioned distally from the proximal end 13 of the connector. The distal end 14 of the connector is in FIG. 2 illustrated as extending into a lumen 21 of a coupling 20. The coupling has a proximal end 22, at an opening 23 in a lid 24, which only is partly illustrated in FIG. 2. In other examples, the opening 23 is not in a lid 24 of a container, but rather in a wall (side or top wall) of a container, which is not functioning as a lid. The coupling 20 may be integral with or coupled to a dip-tube (not illustrated), which extends below the water surface in the container and may extend all the way to the bottom of the container. A sealing 25 may be provided between the connector 10 and a sidewall 26 of the coupling 20, the sidewall surrounding the axial lumen 21. In the illustrated example, the sealing 25 is in the form of a gasket, however other examples may relate to sealings in the form of a cooperation between bulges, protrusions, in one part and indentation or grooves in another part-or between two bulges or protrusions on opposite parts.

The connector may be provided with a tube interface 12 at a proximal portion of the connector, which is configured for attaching a tube to the connector—see FIG. 1. In the illustrated example of FIGS. 2 to 4, the tube interface 12 is configured for permanently attaching a tube to the connector by welding or by use of an adhesive. However, in other examples, the tube interface may be in the form of a tube-coupling (not shown) for detachable coupling of a tube to the connector. The connector is, in the illustrated example, provided with ribs 15 functioning as a gripping aid for increasing the friction between the fingers and the connector, when the connector is to be removed/detached from the container. The connector has a proximal end 13 and a distal end 14 and an axial lumen 16 extending therebetween.

The coupling 20 is in the illustrated example provided with a hole 27 in a sidewall 26 of the coupling. The hole is configured for providing room for the gripper 31 of the toggle arm 30, such that the gripper 31 can catch and hold the flange 11 of the connector as described in the following.

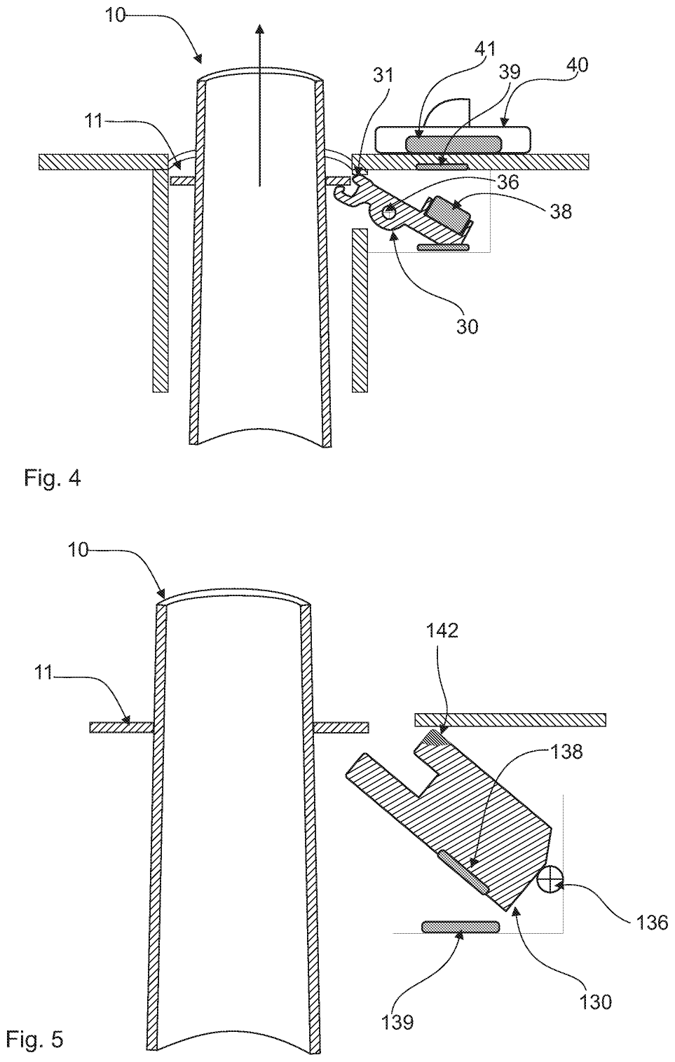

The toggle arm 30 with a gripper 31 forms part of the coupling 20 in the illustrated example. The gripper 31 has the form of a jaw 32 with an upper part 33 and a lower part 34, the upper part 33 extending shorter in the proximal direction than the lower part. Thus, in a first position of the toggle arm, when the toggle arm 30 is at an angle with respect to the direction of the axial lumen 16 of the connector, the jaw extends angularly upwards and allows the flange 11 to be caught in the mouth 35 of the jaw 32—this is the position illustrated in FIG. 2. The toggle arm is attached to the coupling (which is attached to or integral with the container) such that it can rotate (or tilt) around a tilting-axis 36. In the example illustrated in FIGS. 2 to 4 the tilting-axis 36 is located approximate at a middle portion of the toggle arm between the proximal end with the gripper and the distal end. A first hold 38 is at a distal portion of the toggle arm, on (or at least close to) the upper side of the toggle arm and configured for being in a magnetic hold position, when the toggle arm is approximately horizontal (see FIG. 3). The first hold 38 is configured for being out of magnetic hold position, when the toggle arm is tilted as illustrated in FIGS. 2 and 4. In this position, the toggle arm is at an angle to the direction of the axial lumen of the connector.

When the user inserts the connector 10 into the opening of the container and pushes the connector downwards, the flange 11 of the connector will get into contact with the lower part 34 of the jaw and cause the toggle arm 30 to rotate anti-clockwise in the figure around the tilting axis 36. When the toggle arm 30 reaches a horizontal position (FIG. 3) in which the toggle arm is perpendicular to the axial direction of the connector, the first hold 38 will be in magnetic hold position due to a second hold 39 positioned in the lid. The first and second hold 38, 39 have to be poled such that the first hold is attracted to the second hold. For example, if the first hold is a negatively charged magnetic material, then the second hold is advantageously a positively charged magnet or alternatively, a magnetic material. Oppositely to this, if the first hold is a positively charged magnetic material, the second hold is advantageously a negatively charged magnetic material or alternatively, a magnetic material. In this position, the toggle arm 30 will be in a first position and the connector 10 will be in the first connected position with respect to the coupling, due to the flange 12 being caught in the mouth 35 of the jaw in a position, where the mouth extends in the same direction as the flange, thus preventing the flange (and thereby the connector 10) from moving upwards or downwards. To release the connector, a user moves a slider 40 with a third magnet 41 over the container, until the third magnet 41 is in a position, where it can repulse the first hold 38 from the second hold 39. This means that the third magnet 41 must be charged similar to the first hold, such that e.g. both the first and third magnets are negatively charged. Furthermore, it is an advantage if the third magnet 41 and the first magnet between them have a stronger magnetic repulsive force than the magnetic attraction force between the first and second magnets 38, 39. The coupling may include a release hold 42, which assists in keeping the toggle arm at an angled position by providing attraction between the release hold 42 and the first hold 38. This means that if both the release hold and the first hold are in the form of magnets, they have to be oppositely poled. Alternatively, one of them is a magnet and the other one is in the form of a magnetic material.

FIGS. 5 to 7 illustrate an example of a connector as described herein.

This example differs from the example in FIGS. 2 to 4 in that the toggle arm is different and that the position of the first and second hold differs from the position in the example of FIGS. 2 to 4. In the example of FIGS. 5 to 7, the axially extending of the coupling is not illustrated, but is contemplated to be similar to the lumen illustrated in relation to FIGS. 2 to 4.

The toggle arm 130 in FIGS. 5 to 7 is attached to the container at a tilting axis 136, which is located at a distal end of the toggle arm. The first hold 138 is positioned at a proximal portion of the toggle arm and on (or at least close to) the lower side of the toggle arm. The first hold 138 is configured to be in magnetic hold position by attraction to a second hold 139, which is positioned below the toggle arm. As described in the example of FIGS. 2 to 4, also in this example in FIGS. 5 to 7, either the first hold 138 or the second hold 139 will include a magnet and the other one either an oppositely poled magnet or a magnetic material.

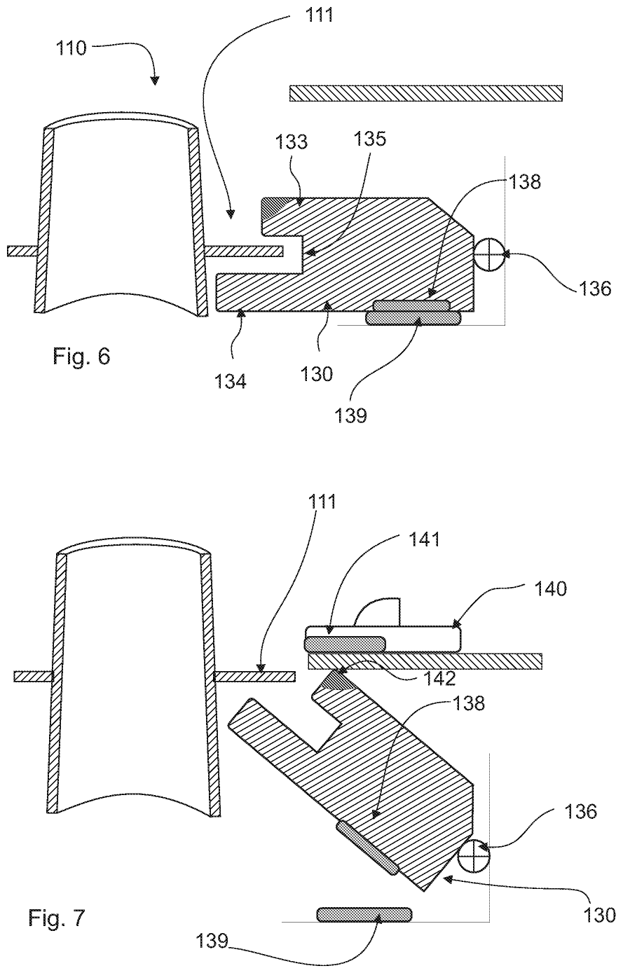

In FIG. 5, the connector is in a second disconnected position, the flange can move freely upwards. When the user wants to connect the connector to the container, the user simply pushes the connector downwards, until it reaches the position illustrated in FIG. 6. In this position, the connector is in the first connected position, because the flange 111 is gripped in the mouth 135 of the toggle arm and is prevented from moving upwards.

FIG. 7 illustrates a position, in which a slider 140 is positioned over the toggle arm 130. The slider has a third magnet 141, which attracts a release hold 142 at a proximal end of the toggle arm 130. The release hold is either a magnetic material or a magnet, which is oppositely poled compared to the third magnet, such that the third magnet 141 is able to attract the release hold. The attraction between these two parts will get the toggle arm to tilt towards the position illustrated in FIG. 7 and thus release the connector flange 111 from the grip of the mouth 135 of the toggle arm.

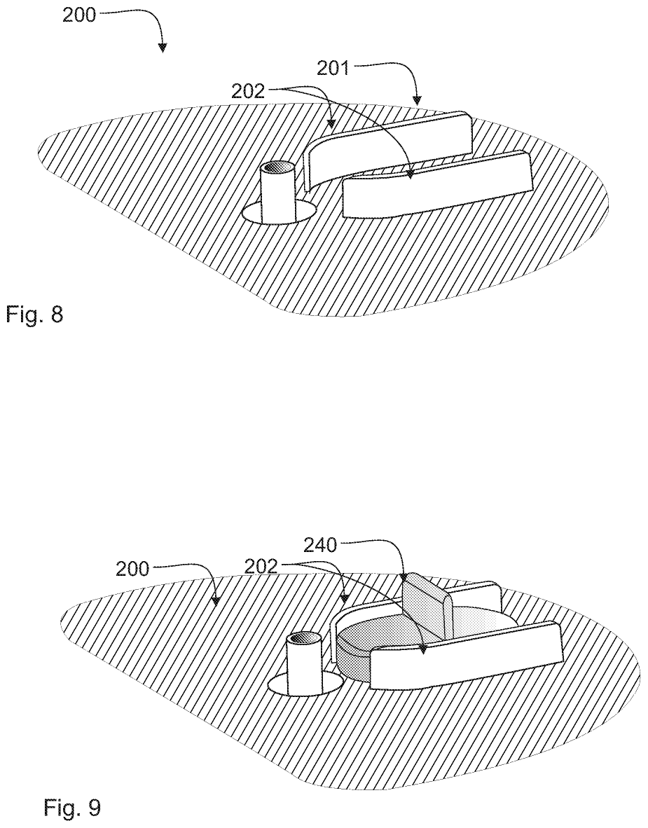

FIGS. 8 and 9 illustrate an example of a guide for the slider. The figures illustrate a portion of a surface 200 of a container, for example the top-surface. On this surface 200, the container may be provided with a guide 201 for the slider 240. The guide 201, may consist of two upstanding ridges 202 as in the illustrated example, however, only one ridge may also be contemplated. When the user wants to position the slider 240 correctly, the user simply moves the slider 240 towards the guide 201. The guide 201 assists in positioning the slider 240 in an optimal position so that a third magnet on the slider 240 is positioned in an optimal position to provide a release function of the toggle arm. This means that the toggle arm is tilted to an angled position, in which the flange on the connector is released from being caught in the mouth of the toggle arm.

Embodiments, and features of the various exemplary embodiments described in this application, may be combined with each other (“mixed and matched”), unless specifically noted otherwise.

1. An anal or stomal irrigation system comprising:
a container configured for containing irrigation liquid;
a connector configured for connecting a tube to the container, a coupling formed in the container, with the connector attachable to the coupling to provide a first connected position, and removable from the coupling to provide a second disconnected position; and,
a toggle arm comprising a first end portion having a gripper adapted to engage with and capture a flange on the connector and a second end portion comprising one of a magnet and magnetic material;
wherein, when the connector is inserted into the coupling, the flange of the connector engages the gripper of the toggle arm, rotationally displacing the toggle arm and capturing the flange within the gripper and engaging the second end portion of the toggle arm into magnetic engagement with a lid of the container.

2. The system as claimed in claim 1, wherein the second end portion of the toggle arm comprises the magnet having a magnetically poled material and the lid of the container comprises the magnetic material.

3. The system as claimed in claim 1, wherein the lid of the container comprises the magnet having a magnetically poled material and the second end portion of the toggle arm comprises a magnetic material.

4. The system as claimed in claim 1, wherein the second end portion of the toggle arm and the lid of the container each comprise a magnet, and the magnet of the toggle arm is oppositely poled from the magnet of the lid.

5. The system as claimed in claim 2, further comprising a third magnet positioned in a slider that is configured to be slid over a surface of the container to release the magnetic hold between the toggle arm and the lid.

6. The system as claimed in claim 5, wherein, when the slider is slid over the surface of the container, the third magnet magnetically repulses the magnet of the toggle arm to release the gripper from the flange of the connector.

7. (canceled)

8. The system as claimed in claim 1, wherein the container comprises a guide formed as a depression integrated into a wall of the container, and the slider is disposed within the guide.

9. (canceled)

10. (canceled)

11. (canceled)

12. (canceled)

13. The system as claimed in claim 1, wherein the toggle arm is configured for rotating about a rotation point located approximately at a middle of the toggle arm.

14. (canceled)

15. The system as claimed in claim 1, further comprising a seal provided at an interface between the connector and the coupling.

16. (canceled)

17. The system as claimed in claim 15, wherein the seal is a radially outwards extending bulge on the connector that is adapted to engage with a radially inwards extending protrusion on the coupling.

18. (canceled)

19. The system as claimed in claim 1, wherein the container comprises a dip-tube configured for providing flow of liquid from a bottom portion of the container to through a lumen of the coupling.

20. The system as claimed in claim 19, wherein the dip-tube extends to a bottom of an interior of the container.

21. The system as claimed in claim 19, wherein the dip-tube is integral with the coupling.

22. (canceled)

23. The system as claimed in claim 1, wherein the toggle arm extends perpendicularly to the connector when the toggle arm is magnetically engaged with the lid of the container.

24. The system as claimed in an claim 1, wherein the container is configured for being pressurized when the toggle arm is magnetically engaged with the lid of the container.

25. The system as claimed in claim 1, further comprising a pump configured to displace the liquid from the container out through the connector and the coupling.

26. The system as claimed in claim 25, wherein the pump is an electric pump.

27. The system as claimed in claim 25, wherein the pump is a manual pump.

28. (canceled)

29. The system as claimed in claim 25, wherein the pump is an air pump configured to pump air into the container to pressurize the container and thereby displace the liquid from the container.

30. (canceled)

31. (canceled)

32. (canceled)

33. (canceled)

34. (canceled)

35. (canceled)
